(19) United States
(12) Patent Application Publication
(10) Pub. No.: US 2016/0361251 A1
(43) Pub. Date: Dec. 15, 2016

(54) NOVEL PROCESS FOR PRODUCING TRANSMUCOSAL PHARMACEUTICAL FORMULATIONS AND FORMULATIONS THUS OBTAINED

(71) Applicant: Clemann Group, Dijon (FR)

(72) Inventor: Patrice BINAY, Couchey (FR)

(73) Assignee: CLEMANN GROUP, Dijon (FR)

(21) Appl. No.: 15/104,028

(22) PCT Filed: Dec. 10, 2014
(86) PCT No.: PCT/FR2014/000265

§ 371 Date: Jun. 13, 2016

(30) Foreign Application Priority Data
Dec. 11, 2013 (FR) 13/02904

(51) Int. Cl. A61K 9/00; A61K 9/20; A61K 9/16; A61K 39/36; A61K 31/137

(52) U.S. Cl. CPC A61K 9/006; A61K 39/36; A61K 31/137; A61K 9/1623; A61K 9/1641; A61K 9/1682; A61K 9/2095

(57) ABSTRACT

The present invention relates to a novel process for producing transmucosal pharmaceutical formulations and to formulations thus obtained. The process relates to the preparation of grains comprising a thermolabile substance as active ingredient, and consists in dissolving said substance in a ternary mixture composed of two amphiphilic binders, the melting point of which is between 40 and 60° C., and of an aqueous solvent such as, in particular, water, an aqueous-alcoholic solution or a water-soluble solvent. The grains thus obtained comprise a thermolabile active substance, two amphiphilic binders and an inert carrier. Use in obtaining translingual tablets wherein the thermolabile active substance is adrenalin.

The present invention relates to a novel method for manufacturing transmucosal pharmaceutical formulations, as well as to the thereby obtained formulations.

More specifically, the method according to the invention and the obtained formulations relate to thermolabile active ingredients, i.e. which may degrade with heat.

Still more specifically, thermolabile active ingredients entering the formulations obtained by the method according to the invention are selected from the group of hormones, proteins, allergens or triptans, from the moment that these substances are sensitive to heat. These active ingredients are known to be fragile, so that traditional methods for manufacturing pharmaceutical formulations containing them are not suitable since they result in their degradation.

From patent EP 0 553 169 application of a method for granulation as a molten mass is known for the production of granules containing active ingredients at a high dosage, the first step of the method consisting in mechanical mixing of the active ingredient and of a binder having a melting point comprised in the interval from 40° C. to 100° C., preferably from 40° C. to 70° C. In this patent, all the mentioned active ingredients are carboxylic acids or salts of these acids. According to the teaching of this patent, there is no dissolution of the active ingredient in the binder appearing in molten form, but a simple mechanical mixing of said active ingredient and of the aforementioned binder.

The object of the method according to the invention is, oppositely to the aforementioned method, to dissolve the active ingredient in a binder with a low melting point.

A first test was therefore carried out aiming at obtaining dissolution of the active ingredient in a binder wherein the binder was an amphiphilic polymer of the polyethylene glycol stearate type (PEG) 1500, designated hereafter as stearate 1500, and the active ingredient is adrenaline tartrate.

The first heating operation shows the melting of the polymer (endotherm at 50° C.), followed by a plateau up to 116° C.: beyond, a beginning of an endotherm which spreads as far as the melting temperature of adrenaline tartrate at 150° C.

During cooling, a re-solidification exotherm is demonstrated (peak at 22° C.). At the end of the analysis, the sample is all the redder since the temperature was raised during the test; this color corresponds to the degradation of adrenaline into adrenochrome at the end of the test demonstrating degradation by oxidation.

The adrenaline-polymer system is unstable beyond 116° C. The latter has a problem related to the heat sensitivity of adrenaline.

Accordingly, it seems to be difficult to develop a reliable and robust method only using a polymer of the stearate 1500 type. More particularly upon increasing the scale of the method, the contact times allowing dissolution of adrenaline bitartrate into the medium are proportionally increased, even more promoting the formation of adrenochrome.

It thus seem to be essential to carry out dissolution of the active ingredient at a much lower temperature, of the order of 50° C., in order to avoid the degradation of adrenaline.

After diverse unsuccessful attempts, it was decided to dissolve adrenaline in a mixture of two amphiphilic polymers, i.e. stearate 1500 mentioned earlier, and TPGS (polyethylene glycol 1000 ester of d-α-tocopheryl succinate), having a melting point around 40° C. The stearate 1500, the TPGS and the adrenaline bitartrate are all soluble in water (while water is not very soluble in TPGS).

Another possibility consist of passing through the intermediate formation of an aqueous solution in which the polymer and the adrenaline are dissolved. A soluble support of the mannitol type or else insoluble support of the microcrystalline cellulose type (MCC) is added to the aqueous solution. The water contained in this mixture is then removed by a drying technique at a low temperature of the freeze-drying type or via the use of a centrifugal evaporator of the speedvac® type in order not to degrade the adrenaline present. It leads to the formation of a compact white mass which is then milled and then sifted after drying in order to obtain a grain with a homogeneous aspect.

More specifically, the operating procedure is the following:

In a 250 ml flask with a wide aperture provided with magnetic stirring and heated by a water bath, add about 2.50 g of TPGS accurately weighed and heat until complete melting of the polymer (40° C.). Add 50 ml of distilled water at 40° C. with stirring until a homogenous solution of the polymer in water is obtained. Cool the solution at room temperature.

Prepare separately a solution of 2.27 g of adrenaline bitartrate (equivalent to 1.25 g of basic adrenaline) in 5 ml of distilled water.

Add the latter to the polymeric solution and stir for 2 mins in order to obtain a homogenous solution.

Gradually introduce with the spatula and with stirring, 20.23 g of mannitol to the solution of adrenaline-polymer maintained at 45° C.±5° C. Maintain the stirring for an additional 5 mins after complete addition of the mannitol and let it cool at room temperature.

The obtained mixture is then placed in a centrifuge in vacuo overnight. 25 g of a white compact mass are obtained after drying which is then milled and then sifted in order to obtain a homogenous grain.

HPLC analysis is compliant with the adrenaline level i.e. 5.0% w/w (basic equivalent on the dry material). The loss upon drying is as for it comprised between 0.2 and 0.3% for the grains from mannitol.

Preliminary tests both with stearate 1500 and with TPGS gave the possibility of obtaining quite satisfactory results from a qualitative point of view. Indeed, the analyses confirm the absence of degradation of the active ingredient. But, although this method allows production of a grain with satisfactory quality, the method used passes through a relatively long and not very productive drying/cold evaporation step. This is due to the presence of water in a relatively large amount in the mixture. Moreover adrenaline in an aqueous solution is relatively not very stable. It is likely that the increase in scale of such a method implies longer times and thereby the possible formation of adrenochrome.

After diverse unsuccessful tests, dissolving the adrenaline in a ternary mixture TPGS/stearate 1500/Water was contemplated.

The first step consisted of verifying the feasibility of this ternary mixture.

The addition of a reduced amount of water in TPGS, leads to the formation of a highly viscous and heterogenous gel (presence of lumps).

Increasing the temperature of the mixture does not provide any benefit and does not give the possibility of obtaining a homogenous mass. Worst, the gel consisting of TPGS and of water has a <<melting>> point much higher than that of TPGS alone. After several tests in diverse conditions for adding water, this route was abandoned.

On the other hand, TPGS and stearate 1500 are miscible in all proportions. Beyond 45° C., both of these polymers melt and mix together without any difficulty so as to form a homogenous and stable mass.

It was therefore attempted to make use of the additional physical properties of both of these polymers.

A mixture of equal masses of TPGS and of stearate 1500 added with a volume of water leads to the formation of a viscous and white mass, the consistency of which is similar to that of soft soap which cannot be utilized in this condition.

After several unsuccessful tests setting into play variable proportions of both polymers, optimum conditions were determined, giving the possibility of dissolving a volume of water in a mixture consisting of 90% of stearate 1500 and 10% TPGS. After adding a volume of water at 50° C., a homogenous ternary mixture of stearate 1500, of TPGS and of water was obtained. Good results are obtained by using, at the binary mixture stearate 1500/TPGS mixture (reference being made to the masses of the components) between 75% and 95%, preferentially between 80% and 90%, of stearate 1500 and between 5% and 25%, preferentially between 10% and 20% of TPGS.

This ternary mixture when it is brought to 50° C. has a similar aspect and is handled in an identical way with both aforementioned polymers in the molten condition.

The second step consists of producing a solid solution of adrenaline bitartrate in the aforementioned ternary mixture.

As adrenaline bitartrate is a very water-soluble compound, first of all it should be verified whether it is to directly dissolve adrenaline bitartrate in solid form into said ternary mixture.

For this purpose, 10 g of ternary mixture are prepared from 4.50 g of stearate 1500, 500 mg of TPGS and 5 g of water at 50° C. This mixture is maintained at 50° C. with stirring for 5 minutes. 4.54 g of adrenaline bitartrate as a powder (equivalent to 2.5 g of basic adrenaline) are then added. The dissolution is total and rapid. After only a few seconds with stirring, the mixture becomes translucent and homogenous.

Further, a granular support was directly added into this molten mass. The retained supports are mannitol and ProSolv 90.

Mannitol is a soluble support. Nevertheless under the assumption that the previous mixture contained a limited amount of water, it should be verified whether the grain will be able to be handled after drying, which would open the route to obtaining a totally soluble sublingual tablet.

The tests carried out in this perspective give the possibility of obtaining a homogenous mass. In spite of the relatively large amount of mannitol (40.46 g) for 14.54 g of the solution of polymer, water and adrenaline, the whole remains quite tacky. After drying in vacuo at 25° C. for 24 h, we obtained a mass which is no longer tacky. After milling and sifting, a perfectly homogenous grain is obtained.

HPLC analysis of this test is compliant with expectations i.e. 5.0% w/w of adrenaline (basic equivalent on dry material). The loss upon drying is as for it comprised between 0.2 and 0.3% for the grains from mannitol.

Preferentially, the grains obtained according to the method described above and entering the scope of the present invention will comprise between 0.5% and 10% of basic adrenaline equivalent, 35% to 45% of stearate 1500, 2% to 10% of TPGS and 40% to 60% of mannitol.

In parallel, several tests were conducted with an insoluble support based on ProSolv 90. This excipient consists of 98% of MCC (microcrystalline cellulose) and 2% of colloidal silica. It gives the possibility of obtaining much more easily a very homogenous grain and which is not tacky; a drying step identical with the tests based on mannitol was however carried out. The HPLC analysis is also compliant with the adrenaline level, i.e. 5.0% w/w (basic equivalent on dry material). On the other hand, although the macroscopic aspect of the obtained grain is of a more fluid aspect, the latter contains more water. The loss upon drying of these tests is as for it comprised between 1.7 and 6.2% according to the tests and the provided drying times. This is explained by the presence of the colloidal silica contained in this excipient which forms a hydrate. Moreover, the tablets which will be obtained from this formula will not be totally soluble. However this does not compromise the efficiency of the sublingual tablet.

An example will now be given, as a non-limiting example for producing a sublingual tablet obtained from grains obtained earlier, wherein the support is mannitol. The contemplated sublingual tablet comprises 5 mg of adrenaline (basic equivalent) for a tablet from 200 to 300 mg with a disintegration time as short as possible and not exceeding 3 mins.

Various mixtures were made with a Turbula (Type IIC) mixer at 45 revolutions per minute, for 10 mins.

The compression operation is carried out on a standard compressing machine. In order to facilitate the analysis of the compression parameters, a set of punches with a cylindrical geometry, with a diameter of 11.28 mm i.e. a surface of 1 cm², planar and not chamfered, is used. The height of the compression chamber upon filling is set so as to obtain the intended mass of the tablets. The thereby produced and analyzed tablets are of course only prototypes, and they do not have the final geometrical characteristics.

The press is equipped with two sensors allowing measurement of the forces which the powder bed exerts on the upper and lower punches, as well as a sensor for the displacement of the upper punch.

The produced tablets are weighed individually, and their dimensions as well as their diametrical breaking force are measured on a durometer of the Kraemer EL Ektronik HP 97 type.

The mass uniformity test was carried out according to the conditions of the European Pharmacopeia 5^{th} edition (2.9.5). It deals with 10 tablets, produced and individually weighed.

The friability test was carried out on PTF E/ER (Pharmatest) according to the conditions of the European Pharmacopeia 5^{th} edition (2.9.7), on the produced tablets for each of the formulae.

The disintegration test was carried out on PTZ (Pharmatest) according to the conditions of the European Pharmacopeia 5^{th} edition (2.9.1). As the dimensions of the tablets do not exceed 18 mm, the test is conducted on 6 tablets.

The sublingual tablets object of the present invention and obtained from the grains described above should observe a certain number of constraints: hardness, friability, disintegration time, notably cohesion.

Preferentially, ProSolv will be used as an excipient; this product is a multifunctional composite excipient based on 98% of microcrystalline cellulose (MCC) and on 2% of colloidal silica. These pharmacotechnical properties give the possibility in difficult cases of very significantly increasing the cohesion of the system. Moreover, this excipient, if it is not water-soluble, remains nevertheless highly hydrophilic.

A first mixture consisting of 25 g of grain and 15 g of ProSolv 90 gave the possibility of obtaining tablets having interesting pharmacotechnical properties. The system is thus highly cohesive and the average hardness of the tablets obtained from this mixture is comprised between 9.5 and 11 kp depending on the applied consolidation force.

A friability test on 10 tablets shows a friability level of 0.16% which is quite satisfactory. On the other hand, the disintegration time of these tablets is too significant since it is close to 8 mins.

This is why it is advantageous to provide a disintegrating agent, such as for example starch glycolate (Explotab). The latter acts according to 2 complementary disintegration mechanisms, i.e., by capillarity by promoting the penetration of water into the tablet when it is in the mouth and then by swelling thereby promoting rapid destructuration of the tablet.

The addition of Explotab (starch glycolate) in a range comprised between 2 and 4% gave the possibility of obtaining a disintegration time compatible with the set goal, i.e. less than 3 mins. On the other hand, everything being moreover equal, it is known that the addition of a disintegrating agent reduces the hardness of the tablet. It should be noted that the disintegration time very strongly varies for a same mixture composition with the applied consolidation force. Therefore there is an optimum to be determined between the applied consolidation force giving the possibility of obtaining cohesion and satisfactory hardness while allowing a disintegration time compatible with the aforementioned constraints.

As an indication, the following formulation meets the different constraints for a sublingual tablet:

| Composition of the mixture | Mass | % |
|---|---|---|
| Grain | 100 g | 50% |
| ProSolv 90 | 92.8 g | 46.4% |
| Explotab | 5.6 g | 2.8% |
| Saccharine | 1.6 g | 0.8% |
| Total | 200 g | 100% |

The saccharine does not provide any benefit from the pharmacotechnical point of view; on the other hand, it appeared that a tablet made without any saccharine had an unpleasant bitter taste. The amount of added saccharine (0.8%) gave the possibility of masking this bitterness.

By starting with the mixture appearing in the table, a sublingual tablet of about 300 mg was made.

The same method as the one described for adrenaline was again taken up successfully with another heat-sensitive substance, in this case the recombinant allergen rBetv1, a characteristic allergen of trees of the order of fagales and in particular birch.

An example of a preparation of a formulation for which the active ingredient is rBetv1 will now be given.

In an Erlenmeyer provided with magnetic stirring, 1.80 g of stearate 1500 and 200 mg of TPGS are mixed at 50° C. with stirring for 3 minutes in order to form a homogenous and intimate mixture of both polymers. The mixture is then slowly cooled to 40° C. The obtained oil remains fluid and translucent.

1 ml of an aqueous solution of rBetv1 is then prepared at a concentration of 3 mg/ml. Dissolution is rapid and the obtained solution of rBetv1 remains limpid at room temperature.

1 ml of this solution is then introduced into the previous mixture of molten polymer maintained at 40° C. Dissolution is complete and rapid. After a few seconds with stirring, the mixture becomes translucent and homogenous.

Further, 8.0 g of mannitol (Mannogen EZ type for example) are introduced, directly added into this molten mass.

Mannitol is a water-soluble support, nevertheless the previous mixture contains a limited amount of water which does not allow dissolution of the mannitol. A homogeneous mass is thereby obtained, which is dried under reduced pressure at 25° C. for 24 h in order to form a grain which after milling and sifting is perfectly homogenous.

By using this same method with the aforementioned ranges of stearate 1500 and of TPGS, in order to form the binary mixture of amphiphilic binders, it is also possible to end up with formulations containing thermolabile active ingredients other than adrenaline or rBetv1, such as for example triptans which are drugs intended for acute treatments of migraine attacks. Also, the method object of the invention applies to allergens and proteins sensitive to heat.

While remaining within the scope of the present invention, diverse alternatives were able to be tested at the ternary mixture; thus, in addition to both amphiphilic binders, a third component is provided, which, in the given examples relating to adrenaline and rBetv1, is water. In addition to water, an aqueous solvent, such as a hydroalcoholic solution or a water-soluble solvent is perfectly suitable. By water-soluble solvent in the present invention is meant any water-soluble solvent of class III (in the sense of the directives of the “European Medicines Agency” relating to residual solvents).

As for the grains obtained according to the method object of the present invention, it is important not to degrade them during their drying; for this purpose, drying by freeze-drying or drying under reduced pressure at a temperature not exceeding 30° C. gives excellent results.

Further, the aforementioned grains may be compressed as sublingual tablets, as this was described for adrenaline bitartrate.

1. A grain including:
a. a thermolabile active substance selected from the group consisting of hormones, proteins, allergens,
b. two amphiphilic binders for which the melting temperature is comprised between 40 and 60° C.,
c. an inert support.

2. The grain according to claim 1 wherein said hormone is adrenaline.

3. The grain according to claim 1 wherein said allergen is rBetv1.

4. The grain according to claim 1 wherein said amphiphilic binders are stearate 1500 and TPGS.

5. The grain according to claim 1 wherein the inert support is mannitol.

6. The grain according to claim 2 wherein it has the following mass proportions:
a. 0.5% to 10% of basic adrenaline equivalent
b. 35% to 45% of stearate 1500
c. 2% to 10% of TPGS
d. 40% to 60% of mannitol

7. A method for preparing grains according to claim 1, wherein it consists of dissolving said thermolabile active substance in a ternary mixture consisting of said two amphiphilic binders and of an aqueous solvent such as, notably, water, a hydroalcoholic solution or a water-soluble solvent, and then of incorporating the inert support therein.

8. The method according to claim 7, wherein both amphiphilic binders are Stearate 1500 and TPGS.

9. The method according to claim 8 wherein the Stearate 1500 represents between 75% and 95% by mass of said binary mixture and TPGS between 5% and 25%.

10. The method according to claim 9, wherein the Stearate 1500 represents between 80% and 90% by mass of said binary mixture and TPGS between 10% and 20%.

11. The method according to claim 7, wherein said inert support is mannitol.
